(19) United States
(12) Patent Application Publication
(10) Pub. No.: US 2003/0143187 A1
(43) Pub. Date: Jul. 31, 2003

(54) USE OF N-HALAMINE BIOCIDAL POLYMER FOR ODOR CONTROL

(75) Inventors: Shelby D. Worley, Auburn, AL (US); Royall M. Broughton, Auburn, AL (US); Jeffrey F. Williams, Langley, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Auburn University

(21) Appl. No.: 10/295,001

(22) Filed: Nov. 13, 2002

Related U.S. Application Data
(63) Continuation of application No. 09/685,963, filed on Oct. 10, 2000, now abandoned.

(51) Int. Cl. A61K 31/785; A61K 9/70; A01N 1/00

(52) U.S. Cl. 424/78.22; 424/443; 422/37; 442/123

(57) ABSTRACT

The present invention is a method of using a polymeric N-halamine biocidal material as an agent preferably used in conjunction with a matrix material for the reduction of noxious odors caused by the decomposition of organic compounds. In a preferred embodiment of the method according to the present invention, the biocidal N-halamine polymer is provided in a comminuted form and mixed with super absorbent polymer to provide a treatment composition. The treatment composition is emplaced within an article to provide intimate contact between the composition and a microorganism-containing fluid. The fluid is absorbed by the super absorbent polymer thereby providing a surface for the biocidal polymer to contact the

### FIELD OF THE INVENTION

The present invention relates to a method of using a N-halamine biocidal polymer for the purpose of modulating the activity of microorganisms such as bacteria, fungi, and yeasts to cause a reduction of the noxious odors in articles such as disposable diapers, incontinence pads, bandages, sanitary napkins, pantiliners, sponges, animal litter, carpets, fabrics, and air filters.

### BACKGROUND OF THE INVENTION

A variety of microorganisms such as certain bacteria, fungi, and yeasts are capable of aiding the decomposition of bodily fluids such as urine and blood, or in the formation of biofilms, which produce undesirable odors in commercial products. For example, bacteria such as Bacterium ammontiagenes and Proteus mirabilis are known to accentuate the decomposition of urea to form noxious ammonia gas through a urease enzyme catalysis mechanism. See U.S. Pat. No. 5,992,351. If an effective treatment composition for disrupting (inhibiting) the urease action can be found, the undesirable odor created by ammonia gas can be minimized. By effective, the treatment composition should be insoluble to resist migrating to human skin, cost effective to be commercially viable, and stable to withstand long periods of shelf life, such as is suitable for consumer uses.

Conventional treatments have been reported for the reduction of odors caused by microorganisms containing urease. One treatment method involves the use of quaternary ammonium compounds. See J. Pediatrics 39:730 (1951) and U.S. Pat. Nos. 5,981,668 and 6,017,561. Another treatment method involves the use of biocidal biguanides. See J.S.D.C. 113:48 (1997) and Tex. Chem. & Color. 28:28 (1996). Another treatment method involves the use of bacteriostatic boron compounds. See U.S. Pat. Nos. 4,949,672; 5,176,108; 5,944,704; and 5,992,351. A further treatment method involves the use of guanidine salt urease inhibitors. See U.S. Pat. Nos. 4,957,063; 5,097,799. And yet another treatment method involves the use of absorbent carbon particles. See U.S. Pat. No. 5,951,744. And a further treatment method involves the use of cyclodextrin complexing agents. See U.S. Pat. Nos. 5,429,628; 5,714,445. And finally, another treatment method involves the use of urease negative bacteria. See U.S. Pat. Nos. 5,507,250; 5,634,431. While these methods can provide at least partial relief from the noxious odors, there are disadvantages with each method including commercial nonviability, skin sensitivity and possibly respiratory problems for the user.

### SUMMARY OF THE INVENTION

The present invention is a method of using a polymeric N-halamine biocidal material as an agent preferably used in conjunction with a matrix material for the reduction of noxious odors caused by the decomposition of organic compounds.

Suitable biocidal materials suitable for use in the present invention include polymeric cyclic N-halamine biocidal compounds, such as those biocidal polymers including a monomeric repeating unit of one or more structures I, II, III, IV, V, VI, VII, VIII, or IX:

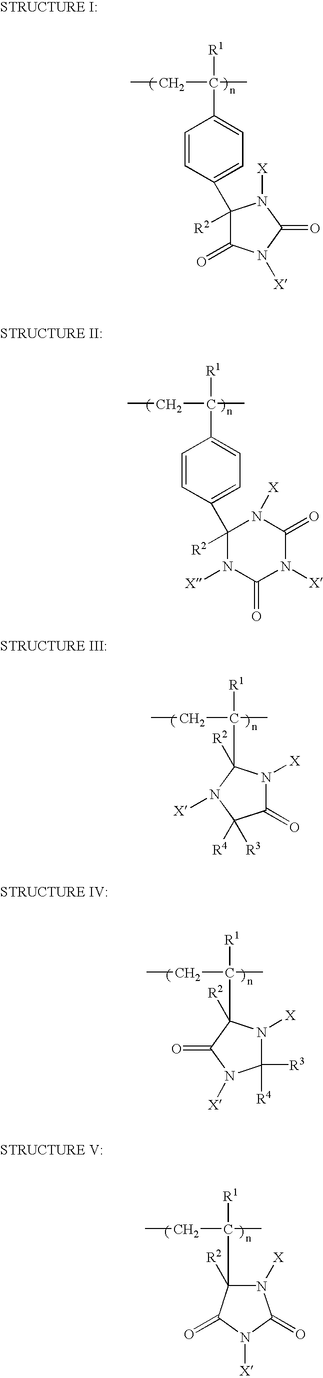

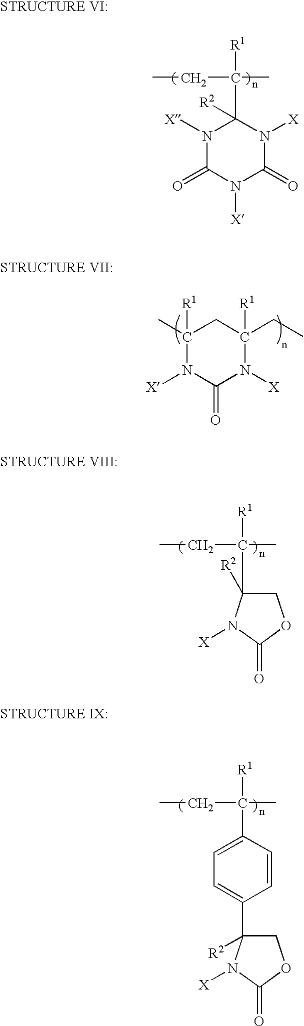

wherein X, X^{I} and X^{II} are independently chlorine, bromine or hydrogen, provided that at least one of X, X^{I} and X^{II} is chlorine or bromine; R¹ is a hydrogen or C₁ to C₄ alkyl; R² is C₁-C₄ alkyl, benzyl or C₁-C₄ alkyl-substituted benzyl; and R³ and R⁴ are independently C₁-C₄ alkyl, phenyl, C₁-C₄ alkyl-substituted phenyl, benzyl or C₁-C₄ alkyl-substituted benzyl, or R³ and R⁴ together form a pentamethylene or tetramethylene moiety.

A preferred biocidal material used in the present invention is poly-1,3-dichloro-5-methyl-5-(4′-vinylphenyl)hydantoin which is an inexpensive derivative of poly-styrene, and which was first described in U.S. Pat. No. 5,490,983, the disclosure of which is hereby expressly incorporated by reference. A monomer unit is represented by the graphical formula:

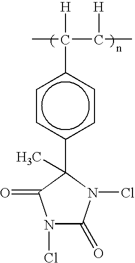

The biocidal properties of poly-1,3-dichloro-5-methyl-5-(4′-vinylphenyl) hydantoin have been recognized to be useful in water filters. See Ind. Eng. Chem. Res. 33:168 (1994); Water Res. Bull. 32:793 (1996); Ind. Eng. Chem. Res. 34:4106 (1995); J. Virolog. Meth. 66:263 (1997) Trends in Polym. Sci. 4:364 (1996); Water Cond. & Pur. 39:96 (1997). But to date, the biocidal polymer has not been applied in a matrix material which provides numerous advantages over the conventional use in water filters. The method of using the biocidal polymer according to the present invention includes providing a quantity of the biocidal polymer, then combining the polymer with a fluid permeable or absorptive matrix material to make a treatment composition. The treatment composition can then be placed in an article or container to bring the biocidal polymer in intimate contact with a microorganism-containing fluid. In a preferred embodiment of the method according to the present invention, the biocidal N-halamine polymer is provided in a comminuted form and mixed with an absorbent polymer, such as super absorbent polymer to provide a treatment composition. The treatment composition is placed within an article to provide intimate contact between the composition and a microorganism-containing fluid, which may be a gas or liquid. The fluid is absorbed by the super absorbent polymer thereby providing a surface for the biocidal polymer to contact the microorganism.

In yet another aspect of the present invention, the biocidal polymer can be coated or imbedded onto the matrix material, such as on a fiber, to provide for control against odor-causing microorganisms.

The method according to the present invention is effective against a broad spectrum of pathogens including Staphylococcus aureus, Pseudomonas aeruginosa, Escherichia coli, Candida albicans, Klebsiella terrigena, and rotavirus, among others, causing large log reductions in contact times of the order of a few seconds in water disinfectant applications. Furthermore, it is effective at pH values at least in the range of about 4.5 to about 9.0 and at temperatures at least in the range of about 4° C. to about 37° C., and it is capable of action even in water containing heavy chlorine demand caused by bioburden.

The biocidal polymer used in the method of the present invention is insoluble in water and organic compounds and will thus not migrate in liquid media. It is stable for long periods of time in dry storage (a shelf life of at least one year at ambient temperature) and can be produced on an industrial scale. Furthermore, all evidence obtained to date suggests that the material is non-toxic and non-sensitizing to humans and animals upon contact.

The present invention thus provides a solid-state biocidal material which is effective against odor-causing microorganisms, insoluble in bodily fluids so as not to migrate to skin surfaces, stable to withstand lengthy shelf life, non-toxic and non- irritating, and cost effective to be commercially viable.

### DETAILED DESCRIPTION OF THE PREFERRED EMBODIMENT

The present invention may be understood more readily by reference to the following detailed description of specific embodiments and the examples included therein.

As used herein, “the biocidal polymer” refers to an insoluble N-halamine polymer, including those described in U.S. Pat. No. 5,490,983, and preferably is poly-1,3-dichloro-5-methyl-5-(4′-vinylphenyl)hydantoin, although this is not meant to be limiting, as any other insoluble N-halamine polymer provides some degree of odor-limiting capacity.

The biocidal polymer to be used according to this invention is preferably mixed in powder or granular form with an absorbent or filler material to provide a treatment composition. The absorbent material preferably is a superabsorbent polymer such as ASAP 2000, sold by the Chemdal Corporation of Palatine, Ill. However, other absorbent materials such as cellulose, diatomaceous earth, cotton, synthetic and natural carpet fibers, cotton or synthetic filter materials can be used. The biocidal polymer preferably comprises a weight percent of about 0.1 to about 5.0 of the treatment composition, and more preferably about 0.5 to about 1.0 for applications involving contact between the treatment composition and human or animal bodily fluids such as occurs in disposable diapers, incontinence pads, bandages, sanitary napkins, pantiliners, sponges, and litter. For applications involving contact between the treatment composition and carpets and other textiles the biocidal polymer will be coated on or embedded in the surfaces of the fibers, granules or other matrix surfaces, at preferably a weight percent of about 0.1 to about 2.0, and more preferably about 0.5 to about 1.0. This will be carried out by using an adhesive or by exposure of heat-softened fibers, granules, etc., to powder streams containing the treatment composition. For air filters, coating techniques or simple embedment of particles of the biocidal polymer into available filter material at preferably a weight percent of about 0.1 to about 2.0, more preferably about 0.5 to about 1.0, can be employed. Since many air filters employ a web of synthetic or cellulosic fibers, the treatment composition resides as a substrate rather than a loose material.

The method according to the present invention will reduce noxious odors to a predetermined level by modulating the level of activity of microorganisms which enhance, through catalytic enzymology, the decomposition of organic matter to ammonia or other noxious materials. The biocidal polymer also will reduce noxious odors on fibers or air filters by microorganisms such as those which cause mildew and molds, as well as those from any liquid or aerosol which might contact the surface of these materials. While not intending to be bound to any particular theory, the mechanism through which the biocidal polymer exerts influence over microorganisms is believed to be a result of surface contact of the microorganism with halogen moieties covalently bound to the hydantoin functional groups of the polymer. The halogen atoms are transferred to the cells of the microorganisms where they cause modulation of activity through a mechanism not completely understood, but probably involving oxidation of essential groups contained within the enzymes comprising the organisms. Halogen moieties can include bromine or chlorine.

Preferably, it is contemplated that a broad variety of absorbent and filler materials can be used in conjunction with the biocidal polymer to provide a treatment composition for reducing noxious odors. One purpose of such materials is to enhance contact of fluids, aerosol particles, and solid contaminants with the treatment composition for sufficient periods of time such that the biocidal polymer particles can affect the odor-causing microorganisms. Matrix materials include, but are not limited to: swellable clays, zeolites, alumina, silica, cellulose, wood pulp, fibers, adhesives, coatings and super absorbent polymers or any combination thereof. The treatment composition according to the present invention can further contain adjuvants such as deodorants, fragrances, pigments, dyes, and any combination thereof for cosmetic purposes. The fluid containing the microorganism is preferably flowed through or absorbed by the resulting matrix.

One advantage of the biocidal polymer of this invention over conventional odor-control technology is that the present invention is a more effective biocide against pathogenic microorganisms encountered in medical applications such as S. aureus and P. aeruginosa than are commercial biocides such as the quaternary ammonium salts. Preferably, the treatment composition can serve a dual function, i.e., modulation of odor-causing microorganisms and of disease-causing pathogens. For this reason the treatment composition of the present invention will have widespread use in hospital settings.

It should be understood that the practice of this invention applies to odors generated by microorganisms in both human and animal fluids as well as to airborne and waterborne microorganisms.

The present invention is more particularly described in the following examples, which is intended as illustrative only since numerous modifications and variations therein will be apparent to those skilled in the art.

### EXAMPLES

### Example-1

### Super Absorbent Polymer (SAP)

Super absorbent polymer (SAP) obtained from Chemdal Corp. (Palatine, Ill.) (ASAP 2000) was mixed with comminuted biocidal polymer, wherein the biocidal polymer was synthesized by the methods outlined in U.S. Pat. No. 5,490,983, herein incorporated by reference, in several ratios of weight percentages with 0% biocidal polymer serving as a control. Mixing was accomplished by manually shaking the SAP and biocidal polymer together in a vial. A 0.25 gram sample of each composition was inoculated with 5.0 milliliters of a challenge suspension containing 10% Proteus mirabilis (6.7×10⁷ CFU/mL) in phosphate buffered water (pH 7) and 90% supplemented human, female, pooled urine (the supplement was 1.25 grams urea per 25 mL urine). All samples were incubated at 37° C. for 6 hours.

An odor test panel including of 13 volunteer nonsmokers evaluated the ammonia odor from the samples after 6 hours of incubation. The volunteers rated the odor on a scale of 0 (no odor) to 10 (strong odor). The averaged results are shown in Table 1. It can be seen in Table 1 that the higher the weight percentage of the biocidal polymer, the lower the average odor rating recorded by the panel.

TABLE 1
Qualitative Odor Test Data for Mixtures of the Biocidal Polymer and
Super Absorbent Polymer (SAP)

| Weight % Biocidal Polymer × 100% | Mean Panel Odor Rating |
|---|---|
| 0 | 8.7 |
| 0.1 | 7.8 |
| 1.0 | 2.7 |
| 5.0 | 2.4 |

### Example-2

### Cellulose Diaper Material

Samples of a commercial disposable diaper material (70% cellulose, 30% polyolefin), each weighing about 1 gram, were slurried in 150 milliliters of distilled water in a blender. Then samples of slurried pulp were mixed with biocidal polymer particles (10 to 50 μm) such that the blends contained 0.5, 1.0, and 2.5 percent by weight biocidal polymer. After further mixing in the blender, each sample was transferred to a sterile beaker where it was diluted with a 100 mL portion of distilled water rinse. Each pulp/biocidal polymer mixture was vacuum filtered to remove the water and dried for 48 hours. The resulting “paper” samples then contained the biocidal polymer at the three weight percentages given above.

Each paper sample as well as a control sample of the original diaper material containing no biocidal polymer was then inoculated with 1.0 mL of a suspension of about 1.0×10⁸ CFU/mL of P. mirabilis mixed with 5.0 mL of supplemented urine solution (1.25 grams urea per 25 mL of human, female, pooled urine) in a sterile 250 mL French square bottle. The bottle was constantly aerated with humidified air with any ammonia being produced swept into the bottom of a 1 liter Wheaton bottle containing 1000 mL of ultra-pure water. The water in the Wheaton bottle was stirred constantly with a vortex of about 2 inches. After contact times of 6, 8, 12 and 24 hours, 100 μL aliquots were removed and subjected to ammonia analyses using a SIGMA Diagnostics Ammonia Procedure (No. 171-UV). It was determined by an in-house sniff panel that a 35 mg/L solution of ammonia is the minimum concentration level which can be detected by the average human nose. This translates to 2 mg/L using the SIGMA reagent kit on water aliquots from the Wheaton bottle, i.e., 2 mg/L as registered by the test kit in this experiment should be detectable by the human nose.

The results from this experiment are given in Table II. The data show that for the control containing no biocidal polymer ammonia detectable by the human nose was present after 6 hours contact of the bacteria with the paper. Even after 8 and 12 hours the level of ammonia was not detectable by the human nose for any of the paper samples containing the biocidal polymer. After 24 hours contact, the level was detectable for 0.5 and 1.0 weight percent samples, although considerably reduced relative to the control sample. A slightly higher concentration of ammonia was detected for the 1.0 weight percent sample than for the 0.5 one. This anomaly was probably due to a greater bioburden or less uniform mixing of the biocidal polymer with the cellulose in the former. No ammonia could be detected for the sample containing at least 2.5 weight percent biocidal polymer at 24 hours contact. It can be concluded that the latter concentration of biocidal polymer completely inactivated the odor-causing bacteria. It is evident that dispersing the biocidal polymer throughout a paper matrix can lead to reduction, and even elimination, of odor caused by P. mirabilis.

TABLE II
Quantative Odor Test Data for Mixtures of Biocidal Polymer and
Cellulose/Polyolefin

| | |
|---|---|
| Weight % | |
| Biocidal | Concentration^{a,b} of Ammonia Detected at: |

| Polymer | 6 hours | 8 hours | 12 hours | 24 hours |
|---|---|---|---|---|
| 0 | 2.5 | 6.0 | 15.4 | 59.1 |
| 0.5 | 0 | 0.42 | 1.3 | 9.5 |
| 1.0 | 0 | 0.63 | 1.7 | 12.1 |
| 2.5 | 0 | 0 | 0 | 0 |

^{a}Concentration in mg/L; 2 mg/L is detectable by the human nose under the conditions of the experiment.
^{b}The data for the ammonia test are accurate to within ±0.5 mg/L.

^{a}Concentration in mg/L; 2 mg/L is detectable by the human nose under the conditions of the experiment. ^{b}The data for the ammonia test are accurate to within ±0.5 mg/L.

While the preferred embodiment of the invention has been described, it will be appreciated that various changes can be made therein without departing from the spirit and scope of the invention.

The embodiments of the invention in which an exclusive property or privilege is claimed are defined as follows:

1. A method of controlling the activity of a microorganism in a fluid using N-halamine polymer, comprising:
providing a quantity of biocidal N-halamine polymer;
combining the polymer with a matrix material to provide a treatment composition; and
contacting the composition with a microorganism-containing fluid to modulate the activity of the microorganism.

2. The method of claim 1, wherein the polymer is an insoluble N- halamine polymer.

3. The method of claim 2, wherein the polymer is poly-1,3-dichloro-5-methyl-5-(4′-vinylphenyl)hydantoin.

4. The method of claim 1, wherein the matrix material is selected from the group consisting of swellable clays, zeolites, alumina, silica, cellulose, wood pulp, fibers, adhesives, coatings, super absorbent polymers, papers and any combination thereof.

5. The method of claim 4, wherein the polymer is coated onto any one of the matrix materials.

6. The method of claim 4, wherein the polymer is imbedded into any one of the matrix materials.

7. The method of claim 1, further comprising mixing the composition with an adjuvant selected from the group consisting of deodorants, fragrances, pigments, dyes, and any combination thereof.

8. The method of claim 1, further comprising:
absorbing-the fluid containing the microorganism onto the matrix material thereby providing the contact between the microorganism with the polymer.

9. The method of claim 1, further comprising emplacing the composition within an article selected from the group consisting of diapers, incontinence pads, bandages, sanitary napkins, pantiliners, sponges, litter, carpets, fabrics, and air filters.

10. The method of claim 1, wherein the polymer comprises an amount of about 0.1 to about 5.0 percent by weight of the composition.

11. The method of claim 8, wherein the polymer comprises an amount of about 0.5 to about 1.0 percent by weight of the composition.

12. The method of claim 1, wherein the microorganism is a bacterium, yeast, fungus, protozoan, virus, mold, or algae.

13. The method of claim 1, wherein the microorganism is a bacterium selected from the group consisting of Bacterium ammoniagenes and Proteus mirabilis.

14. The method of claim 1, wherein the fluid is in the liquid phase.

15. The method of claim 1, wherein the fluid is in the gas phase.

16. The method of claim 1, wherein the microorganism is a pathogen selected from the group consisting of Staphylococcus aureus, Pseudomonas aeruginosa, Escherichia coli, Candida albicans, Klebsiella terrigena, and rotavirus.

17. The method of claim 1, wherein the quantity of biocidal polymer is comminuted.

18. The method of claim 17, wherein the polymer and the matrix material are mixed.

19. The method of claim 1, wherein the modulating activity occurs to urease enzymes in the microorganism.

20. The method of claim 1, wherein the pH of the fluid is about 4.5 to about 9.0.

21. The method of claim 1, wherein the composition is effective at temperatures of about 4° C. to 37° C.

22. The method of claim 1, wherein the polymer has a shelf life of at least one year at ambient temperature.

23. The method of claim 1, wherein the modulating activity controls noxious odors.

24. A method of using N-halamine polymer, as an odor control agent comprising:
providing a quantity of comminuted biocidal N-halamine polymer,
mixing the biocidal polymer with super absorbent polymer to provide a treatment composition; and
emplacing the composition within an article to provide for contact between a microorganism-containing fluid and the composition, wherein the super absorbent polymer absorbs the fluid thereby providing a surface for contact between the microorganism and the biocidal polymer.

25. An article of manufacture for controlling noxious odors, comprising:
a quantity of comminuted biocidal N-halamine polymer; and
a quantity of super absorbent polymer mixed with the biocidal polymer to provide a treatment composition, wherein the composition is emplaced within the article to provide for contact between the composition and a microorganism-containing fluid wherein the super absorbent polymer absorbs the fluid thereby providing a surface for contact between the microorganism and the biocidal polymer.

26. An article of manufacture for controlling noxious odors, comprising:
a quantity of biocidal N-halamine polymer; and
a fiber wherein the biocidal polymer is coated or imbedded onto the fiber to provide for contact between the polymer and an odor-causing microorganism present in a fluid medium.
